(19) United States
(12) Patent Application Publication
(10) Pub. No.: US 2015/0314021 A1
(43) Pub. Date: Nov. 5, 2015

(54) STERILIZATION METHOD COMPRISING STERILIZATION FLUID AND ULTRASONICALLY GERERATED CAVITATION MICROBUBBLES

(71) Applicant: DEVIS TECHNOLOGIES INC., North York (CA)

(72) Inventors: George BOTOS, Oakville (CA); Radu ELIAS, North York (CA); Manfred VORMBAUM, Oakville (CA)

(73) Assignee: DEVIS TECHNOLOGIES INC, North York (CA)

(21) Appl. No.: 14/440,263

(22) PCT Filed: Nov. 19, 2013
(86) PCT No.: PCT/CA2013/050885

§ 371 Date: May 1, 2015

Related U.S. Application Data
(60) Provisional application No. 61/728,715, filed on Nov. 20, 2012.

(51) Int. Cl. A61L 2/025; A61L 2/18

(52) U.S. Cl. CPC A61L 2/025; A61L 2/183; A61L 2/186

(57) ABSTRACT

A sterilization method and apparatus uses ultrasonic vibrations and a sterilant bath, preferably ozone or hydrogen peroxide, for cleaning, disinfecting or sterilizing an article, whereby the ultrasonic vibrations generate cavitation microbubbles for damaging microbiological forms in the bath or on the article. The cavitation microbubbles have a diameter of 1-20 microns, preferably 1-10 microns. The use of cavitation microbubbles makes the method and apparatus more effective against microbiological forms. The cavitation microbubbles are generated at ultrasonic vibration frequencies above 100 k Hz and up to 2 Mhz, preferably 250 k Hz to 2 MHz and most preferably at about 500 k Hz.

### REFERENCE TO RELATED APPLICATIONS

This application claims priority from U.S. Provisional Application Ser. No. 61/728,715, filed Nov. 20, 2012 and entitled ULTRASONIC AND OZONE STERILIZER, the contents of which are incorporated herein by reference.

### FIELD OF THE INVENTION

The present disclosure related to methods and apparatus for the sterilization of articles and in particular to sterilization apparatus and methods using ultrasound in combination with a sterilant.

### BACKGROUND ART

Due to the significant replacement cost, medical, dental, veterinary and similar instruments are commonly reused. In order to reduce the risk of infection, these instruments are typically disinfected and/or sterilized prior to reuse.

Sterilization is commonly understood as the process of killing all microbiological forms. Disinfection is commonly understood as the removal of the majority, or 99.99% to 99.9999% of all microbiological forms. Cleaning is understood as removing all visible debris or material from the surface of an item, for example, blood or other biological material from the surface of a medical instrument.

Generally, sterilization can be performed at elevated temperatures, or at ambient temperatures, for example room temperature. Sterilization at ambient temperatures eliminates the waiting period associated with elevated temperature sterilization during which the sterilized equipment needs to cool down before reuse.

Elevated temperature sterilization is generally carried out in a high temperature autoclave, by subjecting the articles to be sterilized to a combination of high temperature and pressurized steam. Autoclaves are generally directed at handling larger batches of instruments. For smaller practices, it may take a while before enough used equipment is accumulated to process a batch in the autoclave. That would require a large stock of instruments, which is both expensive and may increase the risk of cross-contamination.

Sterilization at ambient temperatures generally involves immersing the articles to be sterilized in an environment that is antagonistic to the survival of microbiological forms. These environments generally contain cold sterilants such as hydrogen peroxide, glutaraldehyde or peracetic acid, which operate at ambient temperatures, such as room temperature.

Ozone dissolved in a liquid such as water, in sufficient concentrations, can also be used as a sterilant. Using ozone as a sterilant has the benefit of leaving no residual toxic chemicals after the sterilization process is completed, since ozone decomposes into oxygen. However, dissolved ozone in a liquid is typically not used on its own for instrument sterilization because of the difficulty of dissolving sufficient ozone in a solution to act as a sterilizer, especially at room temperature. Therefore, an ozone-infused liquid at room temperature may be used for sanitization only, but not for sterilization.

Ultrasonic bath devices are commonly used for the cleaning of objects such as medical instruments by dislodging debris from the surface of the object. In some devices, ultrasonic vibrations are used to dislodge debris and microbiological forms from medical instruments. Other devices further immerse the medical devices in a sterilizing solution. The ultrasonic vibrations and the sterilizing solution then act together to clean and sterilize objects.

Azar, in Ultrsonic Cleaning and Cell Disruption (http://www.megasonics.com/Cavitation.pdf), discloses that bubble size and cavitation energy decrease with increasing ultrasound frequency. Azar discloses that higher frequency ultrasonic vibrations create smaller cavitation bubbles and are therefore more suitable for the removal of submicron particles during cleaning. However, Azar teaches the use of an ultrasonic horn operating at 20-50 kHz for cell disruption and discusses the effect of acoustic microstreaming which may occur during ultrasound treatment and which may increase the chances of a small particle, such as a macromolecule or a suspended cell, into the vicinity of a collapsing bubble.

Louisnard and Gonzales-Garcia (Ultrasound Technologies for Food and Bioprocessing Food Engineering Series 2011, pp 13-64 Acoustic Cavitation, Olivier Louisnard, Jose Gonzalez-Garcia) and Brotchie et al. (Effect of Power and Frequency on Bubble-Size Distributions in Acoustic Cavitation; Adam Brotchie, Franz Grieser, and Muthupandian Ashokkumar*, The American Physical Society, PRL 102, 084302 (2009)) disclose that both the energy content and the size of cavitation bubbles decreases exponentially with higher frequencies and that, although an increase in energy input will increase the bubble size, it does not appear possible to counterbalance the bubble size and energy content decrease by increasing the energy input. Thus, when attempting to maximize the energy content of the cavitation bubble during ultrasound treatment, the use of lower frequency ultrasonic vibrations appears beneficial.

US 2007/0059410 teaches a process for washing and disinfecting foodstuffs, using in combination ozone, carbon dioxide, argon, UV radiation and ultrasound under vacuum. The ultrasound frequency used was 20-100 kHz and ultrasound generated cavitation is identified as the effect responsible for destroying bacterial cell walls. However, due to the operating conditions, the numerous types of disinfecting radiation used and the various disinfecting substances used simultaneously, this process is difficult to operate and requires elaborate equipment associated with significant capital cost. A simplified process using ultrasound at ambient pressures, in separation, or in combination with a single sterilant is not disclosed.

U.S. Pat. No. 7,955,631 teaches a process for washing and sterilizing food products, in particular vegetables. The food products are treated in a first step with ultrasound and ultraviolet radiation in combination and in a subsequent step with an ozone atmosphere and ultraviolet radiation in combination. The ultrasound frequency used was 20-40 kHz. The use of multiple treatment steps, different types of disinfecting radiation and repeated micro-filtration makes this process expensive to operate and requires elaborate equipment associated with significant capital cost. The use of ultrasound simultaneously with ozone is not disclosed.

### SUMMARY OF THE INVENTION

It is now an object of the present disclosure to provide a sterilization method and apparatus which overcomes at least one of the disadvantages of the above prior art methods and apparatus.

In one embodiment, the method and apparatus of the present disclosure uses ultrasonic vibrations and a sterilant, preferably ozone or hydrogen peroxide.

In another embodiment, the sterilization method and apparatus of the present disclosure uses ultrasonic vibrations to generate cavitation microbubbles for damaging microbiological forms. In the present disclosure, the term cavitation microbubbles refers to cavitation bubbles having a diameter of 1-20 microns, preferably 1-10 microns. The use of cavitation microbubbles is theorized to significantly increase the contact area between the cavitation bubbles in the fluid and microbiological forms, the latter generally having a size ranging between 0.1 micron and 20 micron.

In a further embodiment, the cavitation microbubbles are generated at ultrasonic vibration frequencies above those used in prior art devices, in particular frequencies above 100 kHz and up to 2 Mhz, preferably 250 kHz to 2 MHz and most preferably at about 500 kHz.

In a further embodiment, the method and apparatus of the present disclosure provides for the sterilizing, disinfecting, or cleaning of items such as medical instruments, by using ultrasonic or megasonic vibrations to physically damage microbiological forms through the effects of cavitation and a sterilant to then kill the damaged microbiological form in order to sterilize, disinfect, or clean the items. The sterilant is preferably an oxidizing agent such as ozone or hydrogen peroxide and the ultrasonic vibrations are preferably produced in an ozonized water bath at an energy level sufficient to generate cavitation microbubbles.

In one general aspect, a method for sterilizing an article is provided which includes the steps of immersing the article in a fluid bath, the fluid bath containing an oxidizing agent such as ozone or hydrogen peroxide; damaging microbiological forms on the article or in the fluid; and allowing the ozone to penetrate the damaged microbiological forms, thereby killing them; whereby the damaging is achieved by generating cavitation microbubbles in the fluid and near a surface of the article through directing an ultrasonic or megasonic vibration through the fluid bath and to the article.

In another general aspect, an apparatus for the sterilization of an article is provided, which apparatus includes a sterilization chamber for holding a fluid bath containing an oxidizing agent such as ozone or hydrogen peroxide and the article, when immersed in the fluid; and an ultrasonic or megasonic generator for generating in the fluid an ultrasonic or megasonic vibration causing cavitation microbubbles to occur in the ozonated fluid and near the article, the cavitation microbubbles being sufficient to damage microbiological forms which may be present in the fluid bath.

The apparatus preferably further includes an oxidizing agent source for infusion of the oxidizing agent into a fluid to create the oxidizing agent containing fluid.

The ultrasonic or megasonic generator preferably generates frequencies above 100 kHz and up to 2 Mhz, more preferably frequencies of 250 kHz to 2 MHz and most preferably a frequency of about 500 kHz, at energy levels, which create cavitation mirobubbles capable of damaging microbiological forms. The frequencies generated by the generator preferably create cavitation energy levels of 0.2 to 200 J/cm².

The inventors of the method and apparatus of the present disclosure surprisingly discovered that damage to microbiological forms can be achieved by using cavitation microbubbles. Furthermore, the inventors surprisingly found that cavitation microbubbles with sufficient cavitation energy to damage microbiological forms can be created at ultrasound frequencies much higher, and thus at much lower energy contents, than previously believed useful. The inventors discovered that, for maximum disinfection efficiency, the ultrasound frequency can be significantly increased above that commonly used and the energy content of the bubbles reduced until microbubbles of a diameter of 20 micron to 1 micron are generated. Without being bound by this theory, the inventors theorize that increasing the area of contact between the cavitation bubble and the microbiological forms is more important for a reliable damaging of the microbiological forms than energy content. The inventors further theorize that bubble size and energy content per bubble are best balanced to maximize the area of contact between the bubbles and the bacteria, while reducing the bubble size only to the point where each bubble still has just enough energy content to damage the organism upon collapse or implosion of the bubble. That appears to be achieved with cavitation microbubbles.

In view of known ultrasonic disinfection and sterilization methods and apparatus being limited to ultrasonic frequencies of 100 kHz or less and the well known fact that both cavitation bubble size and energy content decreases exponentially with increasing frequency, it was surprising that a very significant reduction in viable microorganism count could be achieved even at frequencies significantly above 100 kHz, even at those more than an order of magnitude higher, which generate exponentially smaller bubbles with much lower energy content. Moreover, it was particularly surprising that those smaller bubbles with lower energy content actually result in much higher reductions in viable microorganism count than those achievable at currently used disinfection frequencies, even microbubbles created at megasonic frequencies (0.5-2 MHz). Thus, the smaller and “weaker” microbubbles have proven to have a higher damaging effect than the larger and more powerful cavitation bubbles generated at currently used frequencies of 20-100 kHz.

### BRIEF DESCRIPTION OF THE DRAWINGS

For a better understanding of the embodiments described herein and to show more clearly how they may be carried into effect, reference will now be made, by way of example only, to the accompanying drawings which show the exemplary embodiments and in which:

FIG. 1 is a flow chart of an exemplary disinfecting method in accordance with the present disclosure;

FIG. 2 is a schematic view illustrating an exemplary embodiment of a basic apparatus in accordance with the present disclosure;

FIG. 3 is a schematic view illustrating an exemplary apparatus in accordance with the present disclosure;

FIG. 4 is a functional view illustrating an example embodiment, where the solid arrows represent the direction of the process flow and the dashed arrow lines represent information/signal flow;

FIGS. 5A and 5B are a detailed flowchart illustrating an example sterilization process.

FIG. 6 is a top down sectional view of an embodiment of the device;

FIG. 7 is a sectional view taken along the line A-A of FIG. 6;

FIG. 8 illustrates the comparative results or antimicrobial efficacy testing with ozone and/or ultrasound;

FIG. 9 illustrates the long term comparative results or antimicrobial efficacy testing with ozone and/or ultrasound; and

FIG. 10 illustrates the comparative results or antimicrobial efficacy testing with hydrogen peroxide and/or ultrasound.

### DETAILED DESCRIPTION OF PREFERRED EMBODIMENTS

It will be appreciated that for simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way, but rather as merely describing the implementation of the various embodiments described herein.

The method and apparatus of the present disclosure generally provides for the sterilizing, disinfecting, or cleaning of articles such as medical instruments, by using in combination ultrasonic vibrations and ozone to sterilize the articles. The sterilization is preferably carried out in a sterilant containing fluid bath, such as an ozonated water bath, or a hydrogen peroxide containing water bath, using ultrasonic vibrations above 100 kHz and up to 2 MHz, preferably 250 kHz to 2 MHz and most preferably about 500 kHz. The ultrasonic or megasonic vibrations are used to physically damage microbiological forms through the effects of cavitation microbubbles, while the sterilant is then used to kill the damaged microbiological forms in order to sterilize, disinfect, or clean the articles.

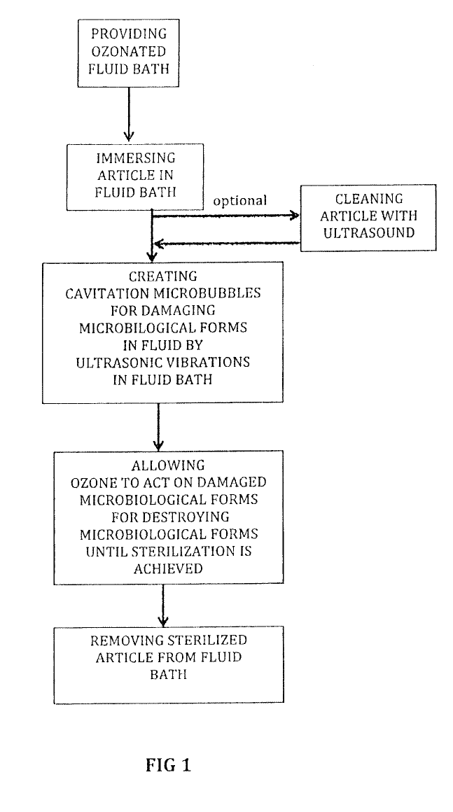

In one exemplary embodiment as illustrated in FIG. 1, a method for sterilizing an article is provided which includes the steps of providing a fluid bath containing an oxidizing agent, such as an ozonated fluid bath, or a hydrogen peroxide containing fluid bath; immersing the article in the fluid bath; creating cavitation mircrobubbles for damaging microbiological forms in the fluid; allowing the ozone to penetrate the damaged microbiological forms thereby killing them; and removing the sterilized article. The damaging is preferably achieved by generating cavitation in the fluid and near a surface of the article through directing an ultrasonic or megasonic vibration through the fluid bath and to the article. The steps of creating and allowing are preferably carried out simultaneously. In an alternate embodiment, the method may include the further optional step of cleaning the article with low frequency ultrasonic vibrations to dislodge larger contamination from the article.

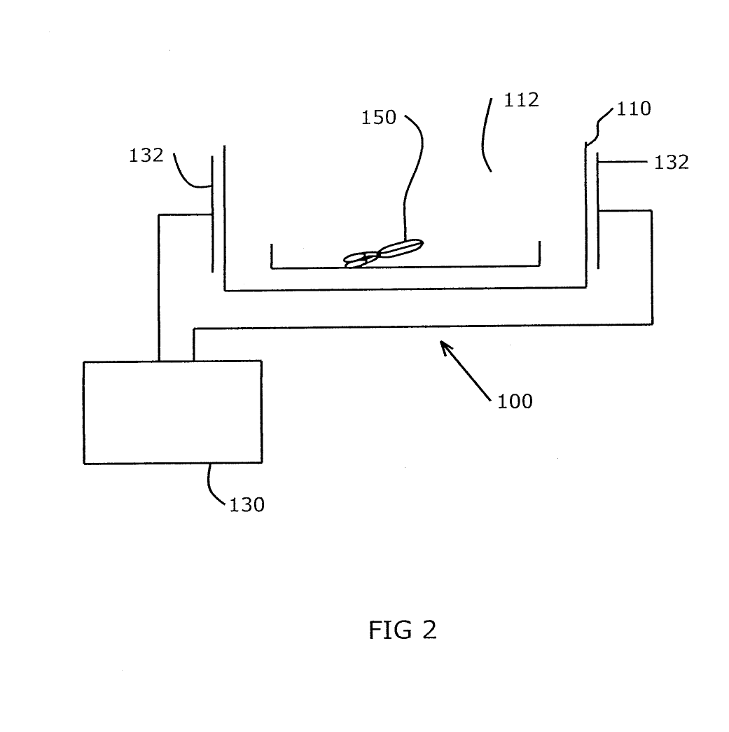

In another exemplary embodiment as schematically illustrated in FIG. 2, an apparatus 100 for the sterilization of an article 150 is provided, which apparatus includes a sterilization chamber 110 for holding a sterilant containing fluid and the article 150, when immersed in the fluid 112; and an ultrasonic or megasonic generator 130 for generating in the fluid 112 an ultrasonic or megasonic vibration causing cavitation in the fluid 112 and near the article 150, whereby the frequency and energy level of the vibrations is selected to generate cavitation microbubbles which are sufficient to damage microbiological forms which may be present in the fluid 112. The apparatus may also include oxidizing agent source (not shown), for example an ozone source 120 for infusion of the oxidizing agent into the fluid 112.

The ultrasonic or megasonic generator 130 preferably generates frequencies above 20 kHz and up to 10 MHz. For sterilization, the generator 130 preferably generates frequencies above 100 kHz and up to 2 MHz, preferably 250 kHz to 2 MHz and most preferably a frequency of about 500 kHz, at energy levels, which create cavitation capable of damaging microbiological forms. The frequencies generated by the generator preferably create cavitation energy levels of 0.2 to 200 J/cm2.

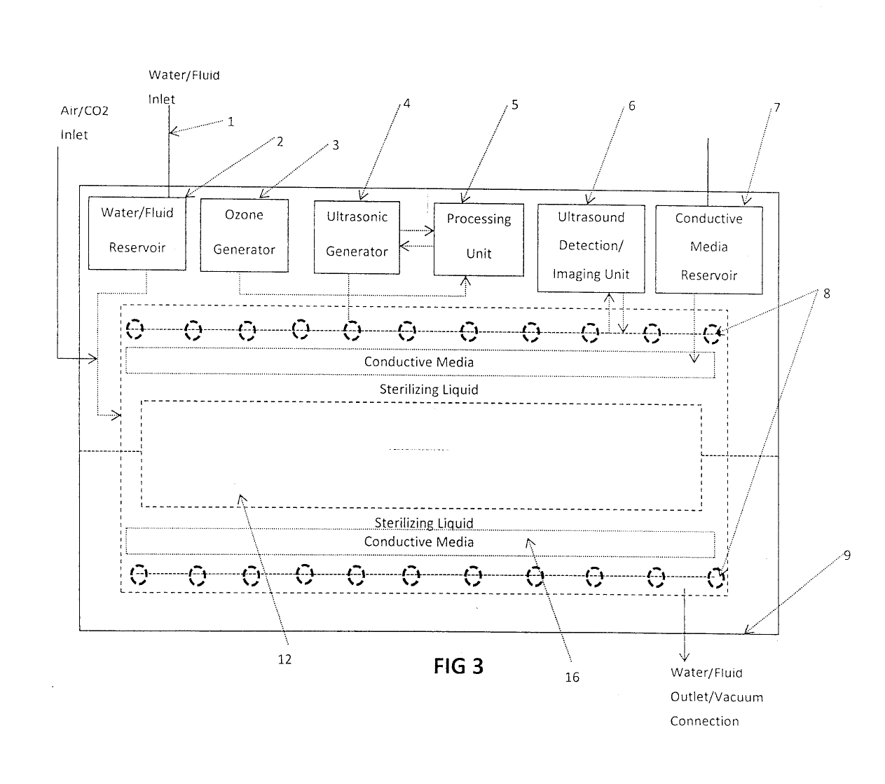

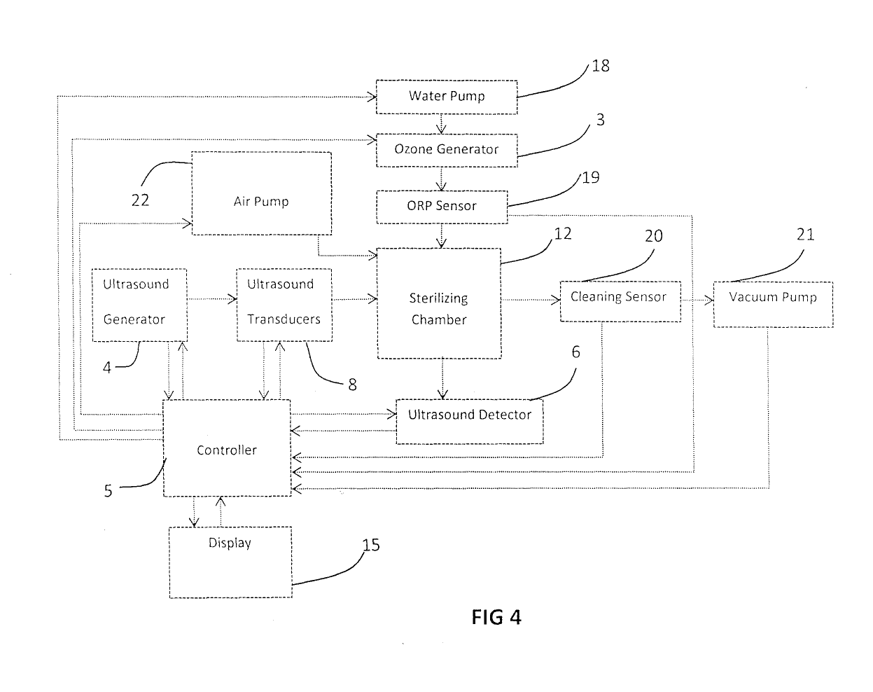

In an exemplary embodiment of an ultrasonic sterilizer apparatus 10 in accordance with the present disclosure as shown in FIGS. 3 and 4, articles, for example medical instruments (not shown) are placed in a sterilizing chamber 12 of the sterilizer 10. The instruments, when placed in the sterilizing chamber 12, are immersed in an ozonated fluid. In one embodiment, an external ozone source may be used to dissolve ozone into a liquid, such as water, to generate the ozonated fluid bath. In the illustrated exemplary embodiment, an ozone generator 3, such as an electronic ozone generator, is integrated into the apparatus 10 for ozonation of the fluid bath. Another type of ozone generator that can be used is a corona discharge ozone generator. Other examples of ozone generators 3 are well known and a skilled technician would understand that alternate ozone generators could be used without departing from the scope of this disclosure. Ozone generators are generally known and need not be described in more detail herein.

The apparatus 10 further includes an ultrasound generator 4 and ultrasonic transducers 8, capable of generating ultrasound vibrations in the fluid bath at frequencies above 100 kHz and up to 2 MHz. Once the ozone has been dissolved into the liquid, ultrasonic vibrations are directed towards the articles to be sterilized by the ultrasound generator 4. The frequency and energy level of the ultrasonic vibrations are chosen to create cavitation in the fluid which is sufficient to damage any microbiological forms that are in the sterilizing chamber 12 either on the article or in the fluid bath. The frequency of the ultrasonic vibrations is chosen to create cavitation microbubbles and the damage caused by the collapse of the cavitation bubbles is sufficient to damage the microbiological forms, so that the ozone in the liquid can sterilize the medical instruments by penetrating the damaged microbiological forms to kill them.

It is known that ultrasonic vibrations, such as a directed, high energy ultrasonic wave can be used to create cavitation that will damage or kill microbiological forms. Cavitation can generate high local temperatures and pressures that can damage or kill microbiological forms. Furthermore, the highly localized temperatures and pressures resulting from cavitation can also denature proteins.

In one embodiment of the apparatus in accordance with the present disclosure, the ultrasonic generator 4 and ultrasonic transducers 8 are capable of generating ultrasonic or megasonic vibrations in the 40 KHz to 10 MHz range.

The ultrasonic generator 4 and ultrasonic transducers 8 can be adjusted to control the frequency of the ultrasonic vibration. For example, during a cleaning step, a lower frequency ultrasonic vibration that generates large cavitation bubbles may be used to remove relatively large pieces of organic matter (for example, blood clots on a scalpel or small pieces of bone on a scraper) from the instruments. During sterilization, a higher frequency ultrasonic vibration is preferably used to generate cavitation bubbles approximating or corresponding to the size of microbiological forms. Those are referred to as cavitation microbubbles herein. For example, a high frequency ultrasonic vibration that generates cavitation bubbles smaller than a single microbiological form can be used to damage the cell wall of the microbiological form.

For example, an ultrasonic vibration in the 400 kHz to 5 MHz range can be generated and used to generate cavitation microbubbles in order to damage or kill microbiological forms. A skilled technician would understand that cavitation at frequencies lower or higher than 400 KHz could be used, for example just above 100 kHz, although cavitation at lower frequencies has a higher risk of also damaging the items to be sterilized. Also, the cavitation bubbles may become too large to damage smaller types of microbiological forms.

The combination of ultrasonic vibration and an ozone-infused fluid can be effectively used to sterilize items such as medical devices. For example, in the case of spores having a defensive outer shell, a room-temperature ozone-infused liquid would have an insufficient ozone concentration to destroy the spores. However, a high frequency ultrasonic vibration above 100 kHz can be used to damage or destroy the cell wall or spore shell, or cause cell lysis. Once the cell has been sufficiently damaged, the ozone in the liquid can penetrate the cell membrane and react with the interior of the spore to destroy the interior of the spore by, for example, denaturing the DNA of the spore. A skilled technician would understand that the use of ultrasonic vibrations causing cavitation microbubbles in combination with an ozone-infused fluid would have the same effect on viruses, bacteria, and any other microbiological forms.

In another example embodiment, the ultrasonic sterilizer is configured to sterilize small quantities of items such as medical instruments. For instance, a dentist may use the sterilizer to clean a set of dental tools associated with a single patient.

In another example embodiment, the ultrasonic sterilizer is portable and can be used in the field. The portable sterilizer may be powered by a portable power supply such as a battery, generator, or fuel cell. This example embodiment could be used by veterinarians working in a rural environment.

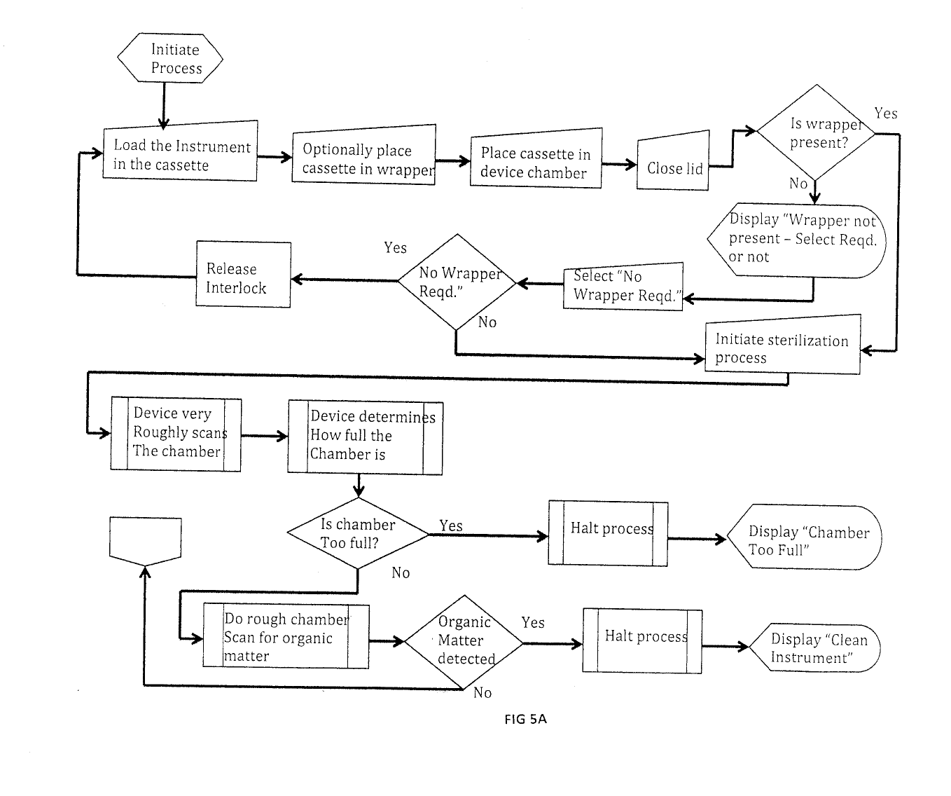

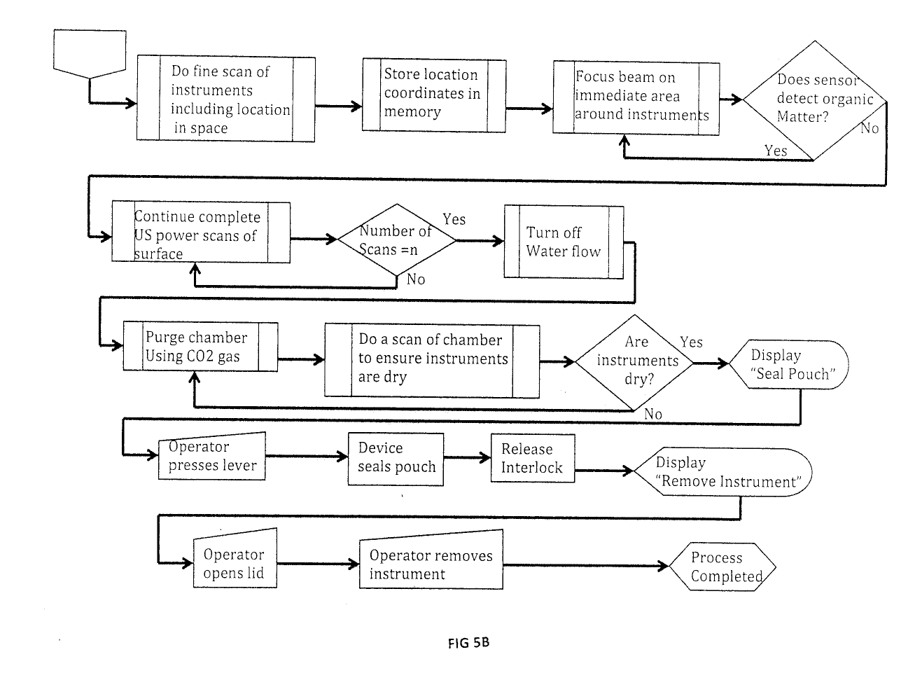

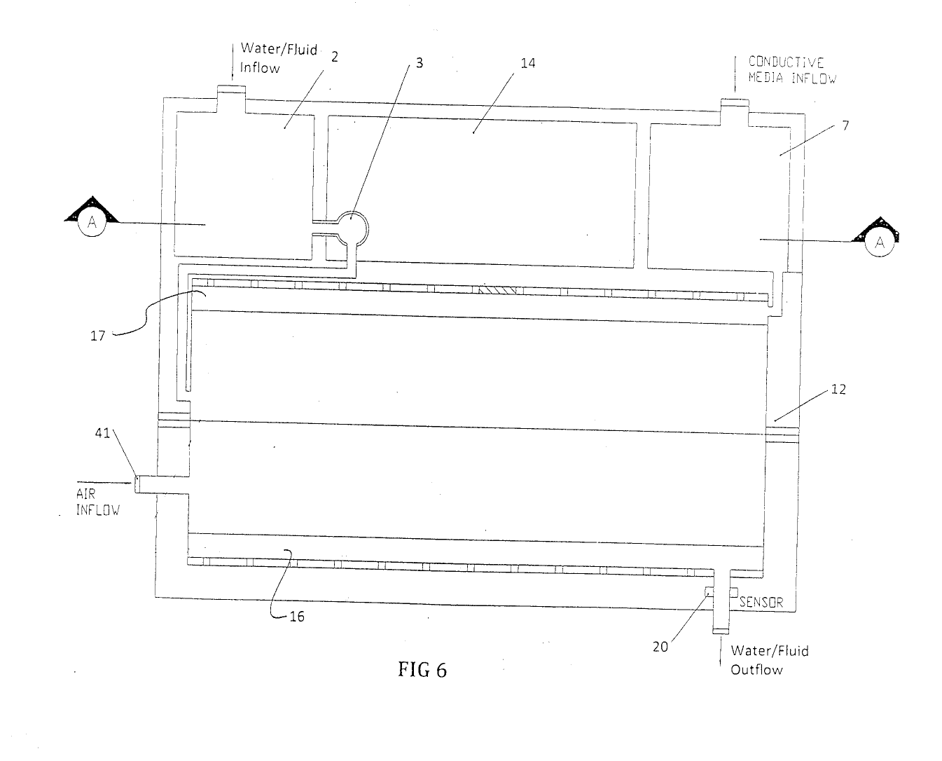

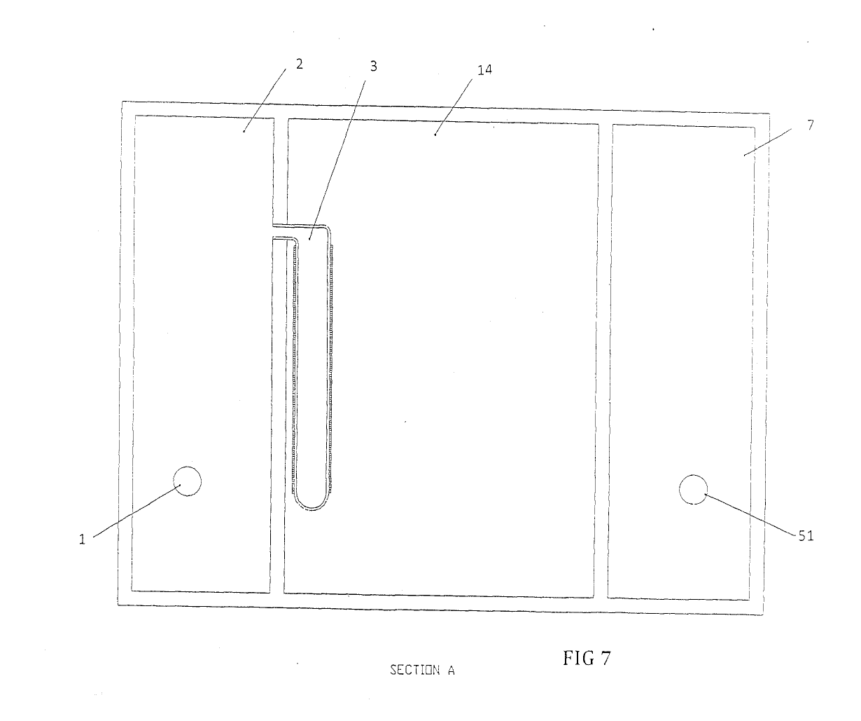

Referring now to FIG. 5, a detailed flowchart illustrating an exemplary method of using the exemplary apparatus is described. The medical instruments to be sterilized are loaded into the sterilizing chamber 12 of the ultrasonic sterilizer. In the example embodiment of FIG. 5, the medical instruments are loaded into a sterilization tray or cassette (not shown) that contains the instruments. This cassette is configured so that it does not interfere with the ultrasonic vibrations being applied to the items to be sterilized. For example, the cassette may be a cage made of thin gauge wire or plastic so that ultrasonic vibrations can pass freely through the cassette.

Other example embodiments may allow the instruments to be loaded directly into the sterilizing chamber 12 or for the instruments to be placed upon a rack contained within the sterilizing chamber 12. A skilled technician would understand that alternate ways of placing instruments in the sterilizing chamber could be used without departing from the scope of this disclosure.

The cassette containing the instruments may optionally be placed in a sterilizing pouch analogous to a wrapper (not shown) for preserving the sterilization of the items when the items are removed from the sterilization chamber. This wrapper may be sealed at the end of the sterilization process so that the sterilized instruments will be protected from contamination. A skilled technician would understand that other methods for sealing the wrapper could be used without departing from the scope of this disclosure. The sterilizing pouch should not impede the flow of ozonated liquid through the cassette or interfere with the ultrasonic vibrations. For example, the sterilizing pouch may be open at both ends.

In an example embodiment where the cassette may be placed in a wrapper, the ultrasonic sterilizer can be configured to detect whether the cassette has been placed in the wrapper. If the cassette has not been placed in the wrapper the sterilizer will indicate, through a user interface 15 such as a lcd display, that the cassette has not been wrapped. The sterilizer may also prevent the user from activating the sterilizing process unless the cassette is in a wrapper.

In some scenarios a wrapper may not be required, so the operator may override the wrapper requirement by interacting with the user interface (for example, a touchscreen, not shown) on the sterilizer. This may be useful when the sterilized items are to be used immediately after sterilization.

In an example embodiment, the volume of the sterilizing chamber 12 is adjustable in order to reduce the volume of fluid required to sterilize the items. This reduced volume also reduces the energy required to generate cavitation. For example, the ultrasonic waves travel a shorter distance in a reduced volume of liquid, thereby reducing the amount of energy lost. The reduction in the amount of energy used is an advantage for the portable embodiment.

After loading the instruments or the cassette into the sterilizing chamber 12, a user may adjust the volume of the sterilizing chamber 12 by re-configuring the walls of the sterilizing device. Alternatively, the sterilizing device may automatically adjust the volume of the sterilizing chamber 12. A skilled technician would understand that alternate means of adjusting the volume of the sterilizing chamber 12 could be used without departing from the scope of this disclosure. For example, FIG. 1 illustrates an example embodiment comprising a volume adjusting means. In this example embodiment, the sterilizing chamber 12 comprises a first side 16 and a second side 17. Each of the first 16 and second 17 sides comprises at least one ultrasonic transducer 8 configured to generate ultrasonic vibrations. In some embodiments, the first 16 and second sides 17 are inwardly adjustable towards the center of the sterilizing chamber 12 so that the volume of the sterilizing chamber 12 is reduced. For example, the first 16 and second 17 sides can be flexible and can be filled and drained of an ultrasonic transmissive medium. This allows the first 16 and second 17 walls to be adjustable towards the center of the sterilizing chamber 12. In this example embodiment, the first 16 and second 17 walls are in fluid communication with a ultrasonic transmissive media reservoir 7. In order to adjust the volume of the sterilization chamber 12, the ultrasonic transmissive media can be transferred to and from the ultrasonic transmissive media reservoir 7 to each of the first 16 and second 17 sides through a conductive media inlet 51. In this example embodiment, a processing unit 5 on the device can adjust the volume of the sterilization chamber 12 based on the size of the cassette. In alternate embodiments, a user may manually adjust the volume of the sterilization chamber 12 by manually transferring ultrasonic conductive media from the ultrasonic conductive media reservoir 7 to the first 11 and second 11 sides using a hand operated pump, for example.

In an example embodiment, ultrasound conductive media such as ultrasound jelly can be used. A skilled technician would understand that other ultrasound conductive media could be used without departing from the scope of this disclosure. For example, any conductive media that, without cavitation, efficiently transmits ultrasonic waves generated by the ultrasonic generator 4 and ultrasonic transducers 8 can be used.

In another example embodiment (not shown), the first and second side walls may be slidably configured in order to reduce the volume of the sterilizing chamber. In this example embodiment, the walls are made of one or more sheets of a solid ultrasonic conductive media. Ultrasonic transducers are then mounted on the first and second side walls so that ultrasonic waves are transmitted through the first and second walls into the fluid. A skilled technician would understand that alternate methods of reducing the volume of the sterilizing chamber, such as by mounting the volume reducing means on the top or bottom walls of the sterilizing chamber 12, could be used without departing from the scope of this disclosure.

Once the sterilizing chamber 12 is loaded with items to be sterilized, the chamber 12 is sealed. The ultrasonic sterilizer may incorporate a locking means (not shown) so that the sterilizing chamber 12 cannot be unsealed until either the sterilization cycle is complete or until a fault condition is detected. Once the sterilizing chamber 12 is sealed, it is then filled with a liquid. In an example embodiment, the ultrasonic sterilizer comprises a water reservoir 2 operatively connected to the sterilization chamber 12. In an alternative embodiment, the ultrasonic sterilizer may be connectable to an external liquid source. In yet another example embodiment, the ultrasonic sterilizer may be connectable to an external liquid source 1 which is then used to fill a reservoir 2 in fluid communication with the sterilization chamber 12. Examples of such liquids include non-toxic liquids such as filtered or distilled water, though a skilled technician would understand that any ozone-infusable fluid, such as hydrogen peroxide, could be used. Preferably a liquid or fluid that also does not interfere with ultrasonic vibrations or waves, such as filtered or distilled water, is used.

The ozone is then produced by the ozone generator 3 and introduced to the fluid so that the fluid becomes infused with ozone. Depending on the embodiment, ozone can be infused into the fluid in the sterilization chamber 12, in the fluid reservoir 2, at the fluid inlet 1, or any combination thereof. In an example embodiment ozone is infused into a room temperature fluid such as water. It is known that the solubility of ozone in water at or near room temperature is approximately 1 to 2 ppm at 15° C. and that ozone at that concentration is inefficient to act as a sole sterilant. As was discussed above, however, the combination of ozone-infused fluid and ultrasonic vibrations for damaging the microbiological forms can be used to sterilize items such as medical instruments.

In an example embodiment the ozone-infused fluid is re-circulated between the sterilization chamber 12 and the fluid reservoir 2. In a preferred embodiment, the ultrasonic sterilizer has a pump (not shown) for circulating the liquid between the fluid reservoir 2 and the sterilization chamber 12. This embodiment, however, may increase the risk of re-contamination as the same fluid is being re-circulated throughout the sterilization cycles. Therefore, filters or systems may be used to clean the fluid and to deal with an excess of ozone.

In another example, in the preferred flow-through embodiment, ozone is introduced into the fluid while the fluid is being introduced into the sterilization chamber 12. For instance, an ozone generator 3 may be placed at or near a fluid inlet 1 so that ozone is infused into the water as it is introduced into the sterilizing chamber 12. In this example embodiment, rather than re-circulating the fluid, a continuous flow of ozonated fluid is provided to the sterilizing chamber 12. Once the items are immersed in the ozone infused fluid, the ultrasonic sterilizer may image the contents of the sterilization chamber 12. Imaging the contents of the sterilization chamber 12 may be performed through various means including visual imaging using cameras, or by ultrasonic signal processing.

In an example embodiment, the at least one ultrasonic transducer 8 on each of the first 16 and second 17 sides forms an ultrasonic transducer array. This ultrasonic transducer array can be used to focus and/or direct ultrasonic waves in the sterilizing chamber 12. In this example embodiment, the individual ultrasonic transducers 8 can emit ultrasonic waves such that the waves are phase matched at a desired location in the sterilizing chamber, thereby focusing the ultrasonic vibration at that location. A skilled technician would understand that alternative means of directing ultrasonic waves, such a lenses or wave guides, can be used to focus the waves without departing from the scope of this disclosure. In an example embodiment, the sterilizer is configured to image the contents of the sterilizing chamber 12 to determine various operating parameters. In this example embodiment, ultrasonic vibrations are sent into the sterilizing chamber 12 and the resultant reflections are collected at the imager (or ultrasound detection unit) 6 and analyzed by the processing unit 5 to determine, among other things, the location of the items to be sterilized or whether any relatively large chunks of organic matter exist. A skilled technician would understand that alternative methods of imaging the sterilizing chamber 12, such as using a camera or radar, could be used without departing from the scope of this disclosure. Alternatively, ultrasonic transceivers may be used instead of transducers so that the array may both transmit and receive ultrasonic waves. In this example embodiment, prior to initializing the sterilization process the ultrasonic sterilizer scans the sterilization chamber 12 to determine whether a user has exceeded the capacity of the device by loading too many items, for example. This is useful because overloading the ultrasonic sterilizer may result in areas in the sterilization chamber 12 that are either shielded from ultrasonic waves, blocked from the flow of ozonated fluid, or both. This can prevent the device from effectively sterilizing, disinfecting, or cleaning the items. In this example embodiment, the imager will scan, using ultrasonic vibrations, the sterilizing chamber 12. The results of the scan will be used to determine whether the items in the sterilization chamber 12 exceed the sterilizing capacity of the ultrasonic sterilizer. If the capacity of the sterilizer has been exceeded, the ultrasonic sterilizer will notify the user through its display means and halt the sterilization process.

The results of the scan may also be used to determine whether any relatively large pieces of organic material (such as blood clots or small pieces of bone or other unwanted organic or inorganic material) exist in the sterilizing chamber 12 or on the items to be sterilized. In this example embodiment, a scan for organic material is performed separately from the scan for determining whether the sterilizing chamber 12 is overfull. Similarly, if large pieces of organic material are detected the ultrasonic sterilizer will notify the user through its display means and halt the sterilization process. In another example embodiment, if the device is being used as a cleaner the ultrasonic sterilizer will proceed with a cleaning cycle.

The results of the scan may also be used to determine the location of items to be sterilized within the sterilizing chamber 12. This information will be used to direct the directable ultrasonic waves to specific locations on, near, or surrounding the items to be sterilized. This information can be stored in a memory store (not shown) such as a hard drive or flash memory so that the location of the items can be retrieved at a later stage in the sterilization process.

In the example provided, each of these scans is performed independently of any other. That is, a capacity scan is performed first, then an organic material scan, and finally a location scan. A skilled technician would understand that changing the order of the scans would not affect the scope of this disclosure. Furthermore, a skilled technician would understand that alternative methods could be employed to determine the above information, such as by a single scan, without departing from the scope of this disclosure.

Once the initial scans have been completed, the sterilization process is initiated. As was discussed above, the sterilization process generally involves damaging microbiological forms using ultrasonic vibrations so that the ozone and any free radicals generated by cavitation in the fluid can penetrate the microbiological forms and destroy the microbiological form by, for example, denaturing the DNA of the microbiological forms.

In an example embodiment, the main sterilization process uses a feedback loop comprising the following steps:

1) detecting whether organic matter or microbiological forms exist in the sterilization chamber 12;

2) applying ultrasonic vibrations to the items to be sterilized so that the microbiological forms are damaged, allowing ozone to penetrate the microbiological form in order to kill the microbiological form;

3) repeating the detecting and applying steps until no organic matter or microbiological forms are detected; and

4) after no organic matter or microbiological forms are detected, repeating the applying and detecting steps several more times as a safety measure.

The step of detecting whether organic matter exists in the sterilization chamber 12 is used to determine whether a next round of ultrasonic vibration needs to be applied to the items to be sterilized. Generally, if organic matter is detected in the sterilization chamber 12 the ultrasonic sterilizer will proceed with another round. These steps will be repeated until no organic matter is detected. Examples of means for detecting organic matter are provided below.

In an example embodiment, the ultrasonic sterilizer has an ion sensor (not shown) for detecting ozone in the liquid. Generally, ozone is consumed when it comes in contact with biological materials such as bacteria, viruses, or spores. This reduces the amount of ozone available for oxidation, which can be measured using an ion sensor. A series of measurements showing stabilized ozone levels would indicate that there is nothing left to oxidize in the sterilization chamber 12.

In this example embodiment an oxidation reduction potential (ORP) sensor 19 is used to determine the ozone content in the sterilization chamber 12. A skilled technician would understand that alternative methods of determining the oxidative potential of a fluid could be used without departing from the scope of this disclosure. For example, a conductivity sensor could be used to determine the change in ionic substances dissolved in the fluid, and thus to indicate the oxidative potential of the fluid.

In another example embodiment, a cleaning sensor 20 such as a turbidity sensor may also be utilized to indicate whether matter is being removed or washed from the sterilization chamber 12. An indication that no matter is being removed or washed from the sterilization chamber 12 can indicate that all microbiological forms have been removed from the sterilization chamber 12. In an example embodiment using a turbidity sensor, the high frequency sonication at cavitation will break down the biological matter present in the solution, which results in a uniformly distributed suspension of the microbiological forms in water. The turbidity of this suspension will increase with the increase of microbiological forms in the solution and can be used in the feedback loop as an indication if material is being removed off the items to be sterilized. Alternatively, a lower limit of the turbidity of the solution can be set that indicates that nothing more can be removed through sonication. The results of this detection step can then be stored in a memory store (not shown) such as a hard drive or flash memory. These results will be used after the ultrasonic vibration step in order to determine whether an additional round of ultrasonic vibration is required. In this example embodiment, the ion sensor and, turbidity sensor are used to detect organic matter or microbiological forms. A skilled technician, however, would understand that alternative means of detecting organic material in a solution could be used without departing from the scope of this disclosure.

If it is determined that organic material exists in the sterilization chamber 12, ultrasonic vibrations will be applied to the items to be sterilized. As was discussed above, a directed ultrasonic vibration can be applied to an area on or near the items to be sterilized. The cavitation created by the ultrasonic vibrations damages the cell walls of microbiological agents, allowing the ozonated fluid to enter the cell. The cavitation may damage the cell walls through the production of heat or free radicals or both. The combination of the ultrasonic vibration, cavitation generated by the ultrasonic vibration, and ozonated fluid, then, sterilizes the items in the sterilization chamber 12.

In an example embodiment, the location of the items to be sterilized is retrieved from the memory store and used by the processor to direct the ultrasonic vibrations. In this example embodiment, the processor will direct the ultrasound generator 4 to generate ultrasonic vibrations to be sent through the one or more ultrasound transducers 8. These ultrasound transducers are configured to fire in such a way so that the ultrasonic vibrations can be directed to specific locations in the sterilization chamber 12. In this example embodiment, since the location of the instruments is known, the ultrasonic vibrations can be directed on or near the items to be sterilized. As was discussed above, alternate means of directing ultrasonic vibrations, such as the use of lenses or wave guides, can also be used without departing from the scope of this disclosure.

In an example embodiment, the detection means is activated again after the ultrasonic vibrations are applied to the items in the sterilization chamber 12. The results from this second detection can then be compared to the results from the first detection as was stored in the memory store. In another example embodiment, the detection means can be activated while the fluid is being drained from the sterilization chamber 12.

The difference between the first and second detection steps provides an indication of the amount of organic matter or microbiological forms remaining in the sterilization chamber 12. These results are then used to determine whether an additional cycle is required to sterilize the device. In an example embodiment, if the readings taken at the ORP sensors before and after the application of ultrasonic vibrations indicate that the oxidation reduction potential has decreased (indicating that organic matter was oxidized), then another cycle will be performed. In this example embodiment, the cycle will be repeated until the oxidation reduction potential before and after the application of ultrasonic vibrations has stabilized (that is, are substantially the same, within a margin of error).

In another example embodiment, an estimate of the minimum number of cycles can be determined prior to initializing the sterilization loop. In an example embodiment, the ultrasonic sterilizer can determine the minimum number of cycles required to sterilize items based on the temperature of the fluid, the number of ultrasonic transducers used in the ultrasonic sterilizer, the power of the ultrasonic transducers used in the ultrasonic sterilizer, and the result of imaging the items in the sterilizer as discussed above. In this example embodiment, the ultrasonic sterilizer will run through at least the minimum number of cycles, and then add cycles depending on the results of the feedback loop as discussed above. In this example embodiment, the processing unit 5 is configured to handle the detection and calculations use by the ultrasonic sterilizer. A skilled technician would understand that alternative methods, such as connecting the sterilizer to a portable computer such as a laptop, or a digital controller, could be used without departing from the scope of this disclosure.

The processing unit 5 and any components sensitive to fluids may be stored in an electronics chamber 14. This electronics chamber is separate from the sterilization chamber 12 so that none of the electronics are exposed to fluids. In an example embodiment, the electronics chamber is in between the sterilization chamber 12 and the exterior housing (not shown) of the ultrasonic sterilizer.

After the detection means determines that there is no organic matter or microbiological forms in the sterilization chamber 12, the ultrasonic sterilizer will repeat the sterilization cycle several more times as a measure of safety. This is to avoid mistakenly indicating that items are sterilized due to an erroneous detection, for example. In an example embodiment, the number of additional cycles performed as a measure of safety may be determined by using the ambient temperature and the power of the ultrasonic transducers. In another example embodiment, the number of additional cycles performed as a measure of safety may be determined by a number of consecutive non-detections of organic matter or microbiological forms.

After the final sterilization cycle has completed the fluid is purged from the sterilization chamber 12. In an example embodiment, the ultrasonic sterilizer has a fluid outlet or vacuum connection 21 in fluid communication between the sterilization chamber 12 and the exterior of the ultrasonic sterilizer. In another example embodiment, the fluid outlet or vacuum connection may be in fluid communication between the sterilization chamber 12 and a second reservoir (not shown) for storing used fluid. This second reservoir can then be manually emptied by the user.

Once the fluid has been purged from the sterilizing chamber 12, a drying means may be used to dry the items. In an example embodiment, carbon dioxide is forced through the sterilization chamber 12 via a CO₂ inlet 41 in order to dry the items to be sterilized. The carbon dioxide can be provided, for example, by a common compressed CO₂ cartridge. The gases can then be purged from the fluid outlet or vacuum connection 21. In another example embodiment, an air pump 22 that is operatively connected to the sterilization chamber 12 can be used to provide dry the items to be sterilized. In this example embodiment, the air can be passed through a scrubber so that air introduced into the sterilization chamber does not contaminate the items to be sterilized. A skilled technician would understand that alternative gases or means of drying (for example, a heater causing any remaining fluid to gassify) could be used without departing from the scope of this disclosure. A humidity sensor (not shown) may be used to verify that the sterilized instruments are dry. This humidity sensor is configured to detect the moisture levels in the sterilization chamber 12. In this example embodiment, the humidity sensor may be located at or near the fluid outlet or vacuum connection 21.

If the cassette 12 was placed in an unsealed wrapper prior to the sterilization process, the wrapper may be sealed after the items are dried. In an example embodiment, a sealing means may be provided for sealing the wrapper prior to removing the cassette from the sterilization chamber 12. A skilled technician would understand that other means of sealing the wrapper, such as by crimping, could be used without departing from the scope of this disclosure. When using a wrapper that effectively seals the cassette from the environment, sealing the cassette allows the sterilized items to be stored while protecting them from contamination.

After the sterilization has completed, the device may be configured to indicate to the user that the sterilization cycle has completed. In an example embodiment, a LED indicating that the cleaning cycle has completed may be provided. In another example embodiment, the status of the sterilization device may be indicated on a touchscreen or similar user interface device. The locking means is also released at the end of the sterilization so that the sterilization chamber 12 can be accessed and the items removed.

In another example embodiment, the ultrasonic sterilizer may be configured with a record keeping means for conforming with record keeping requirements (such as HIPAA compliance) used, for example, in medical centers. This record keeping means may include information regarding the number of sterilization cycles performed, the date, any other data that may be relevant from a record keeping or auditing perspective. In an example embodiment, the record keeping means may be configured to upload this record keeping data to a central data store. In another example embodiment the record keeping means may be configured to provide a physical print out of the relevant data.

### EXAMPLE

The cytotoxic effect of the combined exposure of ultrasound pulses and a fluid bath treated with an oxidizing agent, for example ozonated water, compared to ozonated water alone was investigated in an experimental setup using the bacterial strains Bacillus atrophaeus and Bacillus stearothermophilus, which are considered the “Gold standards” in sterilization verification. The test involved collecting the cell form sterilization verification test strips, activating as per current USP guideline, and exposing the collected cells to various ultrasound frequencies and oxidizing agents. The tests were intended to show that cavitation generated by high frequency ultrasound pulses the high ultrasonic and megasonic range (100 kHz to 2 MHz) can improve the killing of bacteria in combination with oxidizing agents through biomechanical disruption of the bacterial wall. The tests demonstrated the effect of the combined exposure of ultrasound and oxidizing agents on bacterial cell death, compared to the effect of separate exposure to ultrasound and oxidizing agent.

The two bacteria strains Bacillus atrophaeus and Bacillus stearothermophilus were suspended in distilled water. As oxidizing agents and chemical sterilants were used ozonated water (an ozone generator produced ozone at a concentration of 3.7+/−0.5 PPM), hydrogen peroxide (1.5% concentration) and glutaraldehdye (0.2% concentration). The concentrations of the chemical sterilants were selected to produce a noticeable decrease in the number of viable cells, but not at disinfection or sterilization levels. For example, glutaraldehyde as a cold sterilant it is used at 2%, while hydrogen peroxide produces sterilization-level effects at concentrations of over 15%.

The experimental ultrasound setup consisted of a customized exposure chamber, where cells were exposed to ultrasound pulses and/or oxidizing agent. The chamber was made of a modified 3 ml syringe, modified to include two side windows made of an ultrasound permeable polymer. The ultrasound system consisted of a dual arbitrary waveform generator, a power amplifier, a diplexer and an oscilloscope coupled with an ultrasonic transducer positioned in a water tank with deionized water at 20° C. The suspended cells were irradiated with ultrasound at frequencies of 250 kHz, 500 kHz, 1 MHz and 2 MHz. Ultrasound energy levels (150 mV and 300 mV) were chosen to ensure cavitation is achieved during all tests performed.

Comparative testing with exposure for 5 min to ultrasound at 250 kHz, 500 kHz and 1 MHz and using ultrasound and sterilant in separation as well as in combination was conducted and the reduction in viable cell count was determined using standard methodology. The results are summarized in Table 1 below. As is apparent, for a 5 minutes exposure time, ultrasound alone had a much higher cell count reducing effect than any of the chemical agents used. Also the combination of Ozone and Ultrasound showed the highest overall decrease in cell count (the test with ozone at 250 kHz could not be conducted due to failure of the ozone generator).

TABLE 1
Percentage reduction in number of viable
cells upon a 5 minute exposure

| Experiment | 500 kHz | 1 MHz |
|---|---|---|
| Control (normalized) | 100% | 100% |
| US | 96% | 79% |
| Ozone 3.7 ppm | 81% | 71% |
| Ozone + US | 99% | 83% |
| Hydrogen Peroxide 1.5% w/w | 59% | 68% |
| Hydrogen Peroxide + US | 91% | 83% |
| Glutaraldehyde 0.2% | 86% | — |
| Glutaraldehyde + US | 98% | — |

The results show that ultrasound alone has a major effect on cell death, causing a very significant decrease in viable cell counts. The effect of ultrasound alone was tested at 250 kHz, 500 kHz, 1 MHz and 2 MHz at and exposure time of 5 min and at a constant energy level. The results are summarized in Table 2 below, which shows the cell count reduction at each frequency. The effect of ultrasound alone seems to be greatest at 500 kHz, producing the greatest reduction in viable cells for a 5 minute exposure, but good results are obtained for 1 MHz and 2 MHz at which frequencies the cell count reduction is still significantly higher than at 250 kHz.

TABLE 2
Effect of Ultrasound alone
Ultrasound Frequency

| | 0.25 | 0.5 | 1 MHz | 2 MHz |
|---|---|---|---|---|
| US effect (5 min, 300 mV | 50% | 96% | 79% | 84% |

Although conventional ultrasound apparatus for cell disruption are operated at frequencies between 20 and 100 kHz to maximize the cavitation energy per cavitation bubble, the testing results obtained show that significant cell damage can be achieved at much higher frequencies. Without being bound by a particular theory, it appears from the test results that the damage potential of the cavitation bubbles on microbiological forms is more dependent on size, or area of contact, than energy content. Thus, contrary to conventional teaching, using higher frequency (lower cavitation energy) ultrasound appears to be more effective in killing microbiological forms. The spores used in these tests are on average about 1 micron in size (length). The microbubbles formed at 2 MHz are about 1-2 microns in diameter. Thus, if bubble size was the key determining factor in achieving cell damage, that frequency should be most effective. However, as can be seen from Table 2, the most effective frequency for damaging 1-2 micron spores is 500 kHz, which would indicate that although bubble size appears to be the major factor affecting the damage potential of the ultrasound vibrations, the cavitation energy appears to play a role as well. Thus, although higher than expected frequencies have a significant membrane disrupting and killing effect on microbiological forms of 1-10 micron in size, the lower energy of the cavitation and the relatively lower local temperatures produced by the implosion of the cavitation bubble somewhat counteract the increase in contact area. As a result, the most effective bubble size appears to be slightly larger (a few multiples) than the microbiological form, but less than 10 times the size. As can be seen from Table 2, the best membrane disrupting effect was achieved at 500 kHz, which equated, at the energy level used (300 mV) to an approximate bubble size of 0.3 to 1 μm and a significant effect was achieved up to a frequency of 2 Mhz at which the bubble size equals that of the microbiological form while the membrane disrupting effect was reduced to almost half at 250 kHz, where the bubble size is 10 times that of the microbiological form.

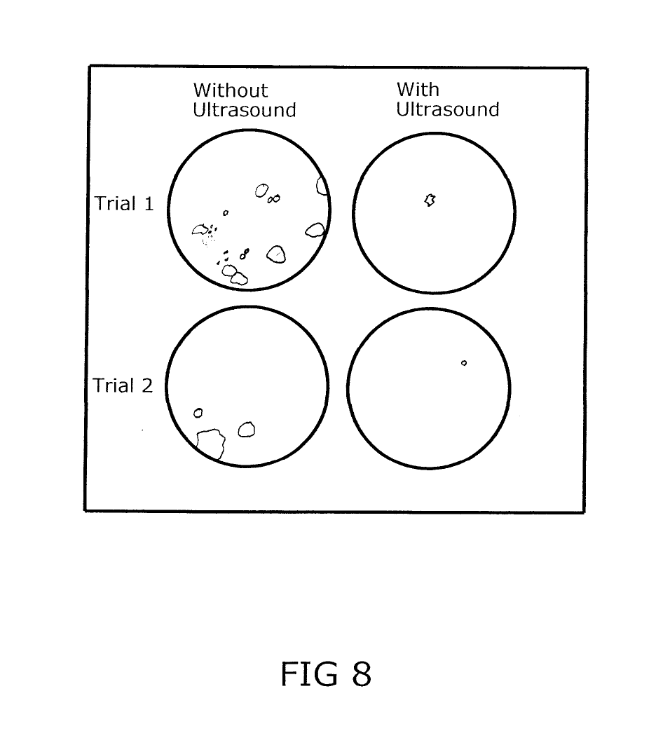

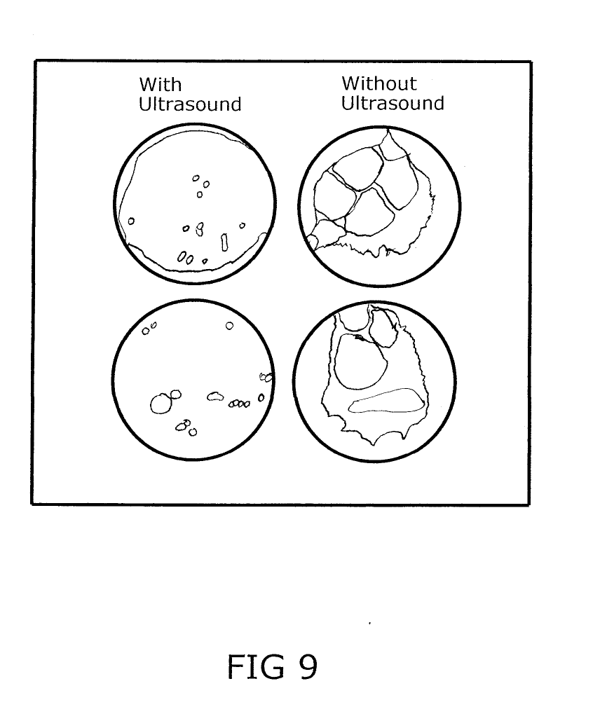

The most interesting results of the experiment were observed in the 500 kHz test using ozone with and without ultrasound. Both for the Ozone only and Ultrasound only samples, bacteria colonies were visible after 24 hours, and the colonies continued to grow over the next 48 hours until the colonies merged. However, with the test using ozone together with ultrasound, the colonies that survived grew very slowly and were barely noticeable after 24 hours, and still very small after 48 hours and even 96 hours (see FIGS. 8 and 9).

The importance of cavitation in the effectiveness of the process, as well as the importance of the exposure time was studied by exposing the cells to two levels of ultrasound energy, both within the ranges known to produce cavitation in this system (150 mV and 300 mV), and exposure of 1 minute and 5 minute, with and without oxidizing agent (hydrogen peroxide). The results are summarized in Table 3 below.

TABLE 3

| | 150 mV | 300 mV |
|---|---|---|
| US 1 min | 41.51% | 44.25% |
| US 5 min | 40.40% | 55.75% |
| US 1 min + H2O2 | 48.93% | 58.91% |
| US 5 min + H2O2 | 77.34% | 70.31% |
| H2O2 1 min | 50.02% | |
| H2O2 5 min | 50.12% | |

The results show that when exposed to ultrasound alone, approximately the same level of reduction in cell count is achieved, regardless of the exposure time and the level of ultrasound energy, which would suggest that most of the damage is produced within the first minute of ultrasound exposure. It also suggest that the cell damage is less dependent of the energy level of the ultrasound, as long as cavitation is produced. Therefore the cell destruction is not directly produced by ultrasound radiation, but by the cavitation microbubbles. Also, given that H2O2 and ultrasound exposure alone respectively produced a lower decrease than the combination, and that there is a significant difference between 1 minute and 5 minutes exposure in combination with hydrogen peroxide, it appears that the mechanism of action is initial damage created by ultrasound cavitation, followed by the oxidative stress produced by the hydrogen peroxide further damaging the cells beyond recovery.

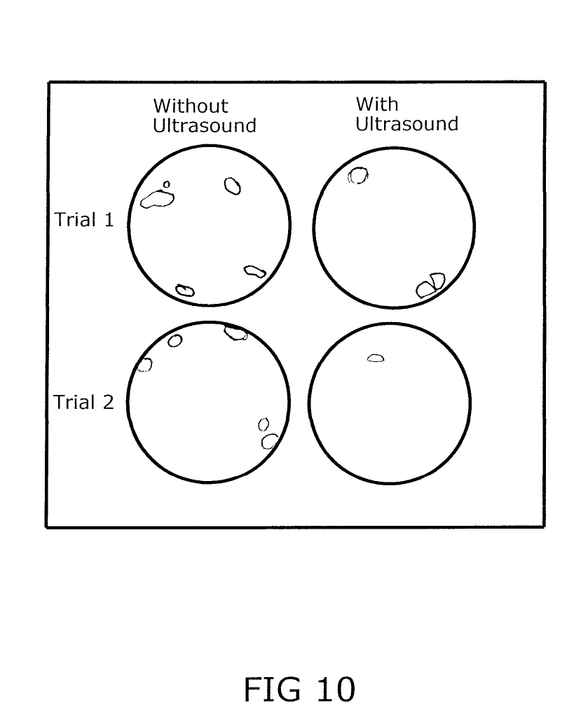

In the parallel tests with hydrogen peroxide and ultrasound, the effect was much less significant (see FIG. 10) than with the test series using ultrasound and ozone. The effect seems to be more evident with ozone than with hydrogen peroxide. The surviving colonies after exposure to ultrasound and hydrogen peroxide seem to develop well even after exposure. The best results overall were achieved with ozone and ultrasound at a frequency of 500 kHz. The very slow growth of the surviving cells suggests that they have suffered significant damage. At the very least their reproductive system appears to have been severely compromised.

Although this disclosure has described and illustrated certain embodiments, it is also to be understood that the apparatus and method described is not restricted to these particular embodiments. Rather, it is understood that all embodiments, which are functional or mechanical equivalents of the specific embodiments and features that have been described and illustrated herein are included. It will be understood that, although various features have been described with respect to one or another of the embodiments, the various features and embodiments may be combined or used in conjunction with other features and embodiments as described and illustrated herein. The above embodiments are not to be taken as indicative of the limits of the invention but rather as exemplary structures which are described by the provided description and claims.

1. A method for sterilizing an article, the method comprising:
providing a sterilant containing fluid bath;
immersing the article in the fluid bath;
creating cavitation microbubbles for damaging microbiological forms in the fluid;
allowing the ozone to penetrate damaged microbiological forms thereby killing them; and
removing the sterilized article.

2. The method of claim 1, wherein the damaging is achieved by generating cavitation microbubbles in the fluid and near a surface of the article through directing an ultrasonic or megasonic vibration through the fluid bath and to the article.

3. The method of claim 2, wherein the sterilant is ozone or hydrogen peroxide.

4. The method of claim 2, wherein the cavitation microbubbles have a diameter of 10 micron to 1 micron.

5. The method of claim 2, wherein a frequency of the ultrasonic or megasonic vibration is more than 100 kHz and up to 2 MHz, for generating cavitation microbubbles having a diameter of 10 micron to 1 micron.

6. The method of claim 1, wherein the steps of creating and allowing are carried out simultaneously.

7. The method of claim 1, comprising the further step of cleaning the article with low frequency ultrasonic vibrations to dislodge larger contamination from the article, prior to the step of creating cavitation microbubbles.

8. The method of claim 7, wherein the low frequency vibrations have a frequency below 100 kHz.

9. A method for sterilizing, disinfecting, or cleaning items as substantially described in the disclosure provided above.

10. An apparatus for sterilizing an article, the apparatus comprising:
a sterilization chamber for holding a sterilant containing fluid and the article when immersed in the ozonated fluid; and
an ultrasonic or megasonic generator configured to generate an ultrasonic or megasonic vibration for generating cavitation microbubbles in the fluid and around the article for damaging microbiological forms in the fluid or on the article.

11. The apparatus of claim 10, wherein the cavitation microbubbles have a diameter of 10 micron to 1 micron.

12. The apparatus of claim 11, wherein the sterilant is ozone or hydrogen peroxide.

13. The apparatus of claim 10, wherein the sterilant is ozone and the apparatus further comprises an ozone source for providing ozone for infusion into a fluid to generate an ozonated fluid.

14. The apparatus of claim 10, wherein the generator generates an ultrasonic or megasonic vibration at a frequency of more than 100 kHz and up to 2 MHz, for generating the cavitation microbubbles in the fluid.

15. (canceled)
